(19) United States
(12) Patent Application Publication
(10) Pub. No.: US 2020/0129400 A1
(43) Pub. Date: Apr. 30, 2020

(54) NAIL COATING SYSTEM

(71) Applicant: Rebecca ISA, Pepper Pike, OH (US)

(72) Inventor: Rebecca ISA, Pepper Pike, OH (US)

(21) Appl. No.: 16/670,302

(22) Filed: Oct. 31, 2019

Related U.S. Application Data
(60) Provisional application No. 62/753,249, filed on Oct. 31, 2018.

(51) Int. Cl. A61K 8/44; A61K 8/33; A61Q 3/02

(52) U.S. Cl. CPC A61K 8/44; A61K 2800/884; A61Q 3/02; A61K 8/33

(57) ABSTRACT

The present technology provides a nail coating system suitable for improving a nail plate. The nail coating system includes an oil-based serum and a base composition. The nail coating system may further comprise a UV/LED composition. In use, the oil-based serum may be applied directly to a nail plate without first cleaning the nail plate with alcohol and/or acetone. The oil-based serum coat may fill in any crevices in the nail plate and restoring sebum and water on the nail plate. After the oil-based serum layer is buffed into the nail or dried, the base composition may be applied. After the base composition dries, the UV/LED composition may be applied and cured. Next, a user may apply a nail polish and/or a top coat to the UV/LED composition. The nail polish and/or top coat layer may be removed with traditional nail polisher remover while still maintaining the original coatings of oil-based serum, a base composition and a UV/LED composition. A new nail polish and/or top coat may thereafter be applied to the UV/LED composition.

### CROSS-REFERENCE TO RELATED APPLICATIONS

The present application claims priority to and the benefit of U.S. Provisional Application No. 62/753,249 entitled “NAIL COATING SYSTEM” filed on Oct. 31, 2018, the disclosure of which is incorporated by reference herein in its entirety.

### FIELD

The present technology relates to compositions and methods for the improvement of common conditions of nails including improved adhesion, improved hydration of the nail, improved nail polish wear, improved strength of nails, rebalancing the sebum and water level of nails, improving the flexibility level in nails, improving the hardness level of nails, the prevention of nail damage, including breakage, fraying, and brittleness, along with the restoration of damaged nail plates.

### BACKGROUND

Natural nail plates, including those in the fingernails and toenails of humans, are comprised of multiple layers of translucent keratin protein. The nail plates are created within a nail matrix at the root of the nail plate and are slowly transported toward the end of the finger or toe, with the entire journey usually taking 4-6 months for fingernails and up to one year for toenails. Keratin, the fibrous structural protein of nails, is also found in human hair and skin. The keratin naturally imparts structure, toughness, durability and flexibility to the nail plates, which ensures resistance to cracking, chipping, breaking and tearing. Nail plates also contain sebum and water to maintain and protect the nail plate. The sebum and water keep the nail plate lubricated and prevent it from drying out and being fragile.

Unfortunately, nail plates can become damaged in everyday activities through exposure to harsh or corrosive chemicals, overexposure to water, mechanical damage, or due to infection or illness, such as certain autoimmune disorders. Some nail plates are also damaged through various manicure and pedicure techniques, e.g., the removal of natural oils, such as sebum and water with alcohol and acetone prior to adding polish to the nails. Further, the nail plates may also suffer from mechanical damage created from overly aggressive and improperly performed manicures, such as artificial nail, acrylic, gel, or poly-gel manicure application and removal procedures. Some people may also inherently have or develop thin or weak nail plates which easily tear, break or peel. The strength and durability of nail plates also normally decrease with advancing age and can vary from person to person based on genetics, diet, and environment. Damaged or weakened nail plates may be more susceptible to infection, excessive staining or discoloration or further continued damage. Also, nail plates can develop grooves of varying depths which run the length of the natural nail plate. These grooves, often incorrectly perceived as ridges, can serve as weak points in the nail plate and act as seeds for initiating longitudinal splits or cracks which can develop in the plate as a result of the significant plate thinning in these grooved areas or provide for weak points in manicures for bubbling or cracking of nail polish.

Increasing or restoring the nail plate's inherent strength, flexibility, and hydration provides increased durability which allows the nail plate to better endure impacts and to resist externally applied physical forces in the form of everyday stresses, strains, polish, etc. There is a balance that must be achieved between strength, flexibility, and durability. An ideal nail is strong enough to resist damage but not brittle, overly flexible, etc. Further, smoothing out the grooves of a nail can allow for improved nail polish wear.

It is therefore desirable to develop a nail plate improvement system that improves strength, durability, and flexibility of nail plates while restoring a nail plate's sebum and water allowing for improved adhesion and wear of nail polish. It is also desirable to develop a nail improvement composition that it easy to apply, yet can provide long-lasting strength and durability to any type of nail plate including those which are already brittle, weak, splitting, or previously damaged. Further, it is desirable to create a nail coating system that can correct and protect the nail plates and nail plate health while also serving as a strong base for the application of cosmetic products to the nail plates.

### SUMMARY

The present technology provides a nail coating system capable of improving nail plate strength, smoothing imperfections in nail plates, restoring sebum and water in a nail plate, improving adhesion of nail lacquer for a colored manicure or pedicure, and resulting in a high quality nail treatment process without the use of chemicals that strip the nail plate such as alcohol, acetone, and/or formaldehyde after preparing a clean nail plate free of nail polish.

In one aspect, the present technology discloses a nail coating system including an oil-based serum and a base composition. The base composition may include a material selected from the group consisting of: acetate, butyl acetate, or derivatives thereof. For example, the base composition may include sucrose acetate isobutyrate. Further, a coating of oil-based serum may be applied directly to a clean nail plate or a nail plate coated in oil and water. The oil-based serum coating may restore sebum and water to the nail plate or secure the oil and water to the nail plate. The oil-based serum may also fill crevices in the nail plate. A coating of base composition may be applied to the oil-based serum coating. The nail coating system may further include a UV/LED composition. The UV/LED composition may be applied to the base composition. The nail coating system may further include a nail polish coating. In an embodiment, the nail polish coating may be applied to the base composition or the UV/LED composition. The nail coating system may further include a top coat. In an embodiment, the top coat may be applied to the base composition, the UV/LED composition, or the nail polish coating. The nail coating system may further include a second UV/LED composition. In an embodiment, the second UV/LED composition may be applied to the first UV/LED composition, the nail polish coating, or the top coat.

In an aspect, the present technology discloses a method of using a nail coating composition including applying an oil-based serum coating to a nail plate, drying the oil-based serum coating or buffing the oil-based serum coating into the nail plate, and applying a base composition coating to the oil-based serum coating. The method may further include applying a UV/LED composition coating to the base composition coating. The method may further comprise applying a nail polish coating to the base composition coating or the UV/LED composition coating. In an embodiment, a top coat coating may be applied to the base composition coating, the UV/LED composition, or the nail polish coating. The method may further comprise applying a second UV/LED composition coating to the first UV/LED composition, the nail polish coating, or the top coat coating. In an embodiment, a nail polish coating may be applied to the base composition coating and may be followed by a top coat coating.

In an embodiment, the method may further comprise removing the nail polish composition coating with a nail polish remover and maintaining the UV/LED composition coating, the base composition coating and the oil-based serum coating. In an embodiment, the method may further comprise further applying a new nail polish coating or top coat to the UV/LED composition coating. The method may further comprise removing the top coat coating and the nail polish composition coating with a nail polish remover and maintaining the UV/LED composition coating, the base composition coating and the oil-based serum coating. In an embodiment, the method may further comprise further applying a new nail polish coating or top coat to the UV/LED composition coating.

In an aspect, the present technology discloses a method of using a nail coating composition including applying an oil-based serum coating to a nail plate with crevices and filling the crevices of the nail plate with the oil-based serum. The method may further include drying the oil-based serum coating or buffing the oil-based serum coating into the nail plate. A base composition may be coated onto the oil-based serum coating and may push the oil-based serum into the nail plate. The method may further comprise applying a UV/LED composition coating to the base composition coating. In an embodiment, the coated nail plate has restored sebum and water levels. In an embodiment, the coated nail plate has improved nail health.

Further, it is desirable to create a nail coating system that allows for easy removal and changing of the cosmetics (nail color) layer without altering integrity and health benefits of the nail coating system below or the necessity to remove said system. This essentially allow end user to change nail polish (traditional solvent based or hybrid solvent and US based) with acetone and non-acetone polish remover without disrupting the nail coating system. It is desirable for the end user for ease of use, flexibility, durability and time saving. This is a completely unique and new possible application.

These and other aspects and embodiments are further understood with reference to the following detailed description.

### BRIEF DESCRIPTION OF THE DRAWINGS

FIG. 1 is a cross-sectional view of the nail coating system on a nail surface;

FIG. 2 is a cross-sectional view of the nail coating system and nail polish coating on a nail surface;

FIG. 3 is a cross-sectional view of the nail coating system, nail polish, and a top coat on a nail surface; and

FIG. 4 is a cross-sectional view of the nail coating system, nail polish, top coat and a second UV/LED coating on a nail surface.

FIG. 5 is a flow-chart detailing various options for using the nail coating system.

The drawings are not to scale unless otherwise noted. The drawings are for the purpose of illustrating aspects and embodiments of the present technology and are not intended to limit the technology to those aspects illustrated therein. Aspects and embodiments of the present technology can be further understood with reference to the following detailed description.

### DETAILED DESCRIPTION

Reference will now be made in detail to embodiments of the present teachings, examples of which are illustrated in the accompanying drawings. It is to be understood that other embodiments may be utilized and structural and functional changes may be made without departing from the scope of the present teachings. Moreover, features of the embodiments may be combined, switched, or altered without departing from the scope of the present teachings, e.g., features of each disclosed embodiment may be combined, switched, or replaced with features of the other disclosed embodiments. As such, the following description is presented by way of illustration and does not limit the various alternatives and modifications that may be made to the illustrated embodiments and still be within the spirit and scope of the present teachings.

As used herein, the words “example” and “exemplary” mean an instance, or illustration. The words “example” or “exemplary” do not indicate a key or preferred aspect or embodiment. The word “or” is intended to be inclusive rather an exclusive, unless context suggests otherwise. As an example, the phrase “A employs B or C,” includes any inclusive permutation (e.g., A employs B; A employs C; or A employs both B and C). As another matter, the articles “a” and “an” are generally intended to mean “one or more” unless context suggests otherwise.

Disclosed is a nail coating system suitable for improving a nail plate. The nail coating system includes an oil-based serum and a base composition. The nail coating system may further comprise a UV/LED composition. In use, the oil-based serum may be applied directly to a nail plate without first cleaning the nail plate with alcohol and/or acetone. The oil-based serum coat may fill in any crevices in the nail plate and restoring sebum and water on the nail plate. After the oil-based serum layer is buffed into the nail or dried, the base composition may be applied. After the base composition dries, the UV/LED composition may be applied and cured. Next, a user may apply a nail polish and/or a top coat to the UV/LED composition. The nail polish and/or top coat layer may be removed with traditional nail polisher remover while still maintaining the original coatings of oil-based serum, a base composition and a UV/LED composition. A new nail polish and/or top coat may thereafter be applied to the UV/LED composition.

The present technology provides a nail coating system capable of improving nail plate strength, protecting the nail from damage, smoothing imperfections in nail plates, restoring sebum and water in a nail plate, improving adhesion of nail lacquer for a colored manicure or pedicure, and resulting in a high quality nail treatment process without the use of chemicals such as alcohol, acetone, and/or formaldehyde.

### Composition

The nail coating system of the present technology includes two components—an oil-based serum component and a base component. Each component may comprise a single component or several different components.

The nail coating system includes an oil-based serum. The oil-based serum is any appropriate type of liquid or gelatinous material. The oil-based serum may be composed of any appropriate materials. In an embodiment, the oil-based serum may include jojoba, argan, rose hip, avocado, olive, horsetail grapeseed, marula, almond, sunflower, coconut, castor, lavender, tea tree, peppermint, sweet orange, or other oils, and/or shea butter, vitamin E gel, beeswax, collagen, keratin, petrolatum, lanolin, Vaseline, aloe vera gel, and the like. The amount of materials may vary based on the batch size and the amount of other components within the oil-based serum.

The oil-based serum may be prepared by combining its component materials in any appropriate manner, e.g., sequentially, all at once, or in various stages. In an embodiment, these components may be formed in-situ. In another embodiment, the components may be preformed materials. After preparation, the oil-based serum component may be stored in any appropriate container, e.g., a standard glass nail polish container.

The nail coating system also includes a base component. The base component is a liquid component. The base component may be composed of any appropriate materials, including but not limited to, acetate, butyl acetate and/or derivatives thereof. The base composition may include sucrose acetate isobutyrate. The amount of these materials may vary based on the batch size and amount of other components within the base component. In an embodiment, the base component may be a traditional nail polish formula containing acetate, butyl acetate and/or derivatives thereof.

The components of the base component may be prepared by combining its component materials in any appropriate manner, e.g., sequentially, all at once, or in various stages. In an embodiment, these components may be formed in-situ. In another embodiment, the components may be preformed materials. After preparation, the base component may be stored in any appropriate container, e.g., a standard glass nail polish container.

The nail coating system also includes a UV/LED component. The UV/LED component is a liquid component and may be a UV/LED activated clear product. The UV/LED component may be composed of any appropriate materials, including but not limited to, acrylate/methacrylate-containing compositions and/or derivatives thereof. The UV/LED component formulation may include reactive monomers, oligomers, cross-linkers, photo-initiators, inhibitors, solvents and, for colored layers, pigments. The amount of these materials may vary based on the batch size and amount of other components within the UV/LED component. In an embodiment, the UV/LED component may be a traditional UV/LED radiation curable nail polish.

The components of the UV/LED component may be prepared by combining its component materials in any appropriate manner, e.g., sequentially, all at once, or in various stages. In an embodiment, these components may be formed in-situ. In another embodiment, the components may be preformed materials. After preparation, the UV/LED component may be stored in any appropriate container, e.g., a standard glass nail polish container.

### Methods of Use

After preparation, the nail coating system may be applied to a nail via any suitable method including, but not limited to, by brush, by sponge, by roller, by spraying, by dipping, by dropping, etc. In an embodiment, the nail coating system may be applied by a standard brush-applicator as commonly used in nail polish application.

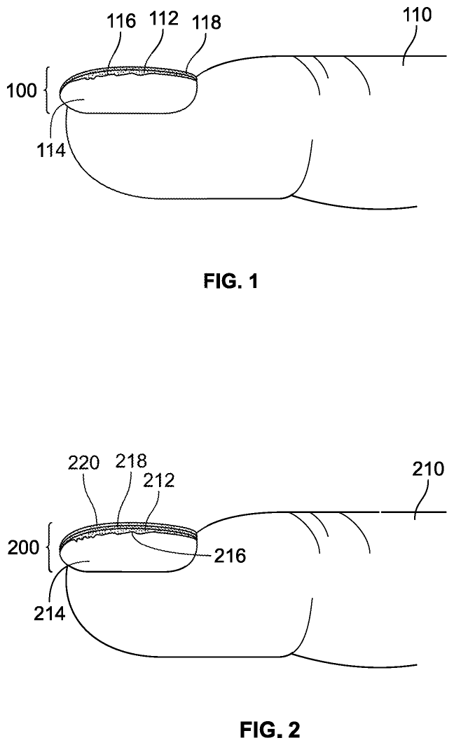

The present technology also includes a method of coating nail plates with the nail coating system 100. As shown FIG. 1, the nail coating composition may be applied to a natural nail of a finger or toe 105. The nail may be free of previous nail products, lotions, and other cosmetics products before the nail coating system is applied. Previously applied nail polish should be removed from the nail by any appropriate means, including, but not limited to acetone or other nail polish remover. The nail does not need to be further cleaned and/or dried with acetone and/or alcohol. The hands/feet, including nails, may be washed with soap and water and dried before applying the nail coating system 100.

The method of applying the nail coating system includes optionally shaking the containers holding the oil-based serum, base composition, and UV/LED component products. The target nails should be cleaned of residual nail polish by any appropriate means, including the use of acetone or other appropriate nail polish removers. Then, the hands and/or feet should be washed with soap and dried, paying attention to remove any old lotions, dirt, debris, etc. from the nails and surrounding skin. The nails may be trimmed with manicure scissors or nail clippers and cuticles and other skin may be maintained as normally done with a manicure and/or pedicure. Then, the oil-based serum 112 may be applied directly to the nail plate 110 and surrounding skin up to the knuckle, including cuticles and under the free edge of the nail. This step is counter-intuitive as in normal manicure/pedicure processes, the nail plate is first cleaned with an alcohol to remove sebum oil and cleaned with acetone to remove water from the nail plate. This step dries out the nail as removing the natural protective sebum and water leaves the nail plate dry and brittle, i.e., vulnerable to further damage.

With the present nail coating system, the oil-based serum 112 should be applied directly to the nail plate that still contains its natural sebum and water, i.e., one that has not been treated with alcohol, acetone, or any other harsh chemical and/or drying agents except as may be incidental during the removal of a prior nail polish. Here, the oil-based serum 112 adheres directly to the nail plate 110, filling in any natural crevices 114 caused by damage from previous nail treatments, including, but not limited to, the application and removal of artificial nails or gel nail polish, or contact damage, or patterns in natural nail growth due to illness or disease. The oil-based serum 112 then creates a smooth nail plate surface, allowing for better adhesion of the next layers of nail product. The oil-based serum 112 is absorbed into the nail, locking in already existing sebum and water, and encouraging the production of additional sebum and water to help correct and restore the nail plate to its natural, healthy state.

The coat of oil-based serum 112 may be applied in any appropriate amount, enough to create a smooth covering over the user's nail plate 110. Drying time of the oil-based serum 112 may vary based on various factors, including, but not limited to, the thickness of the coat of oil-based serum, the conditions and temperature in the location of the nail coating composition process, etc. Next, the oil-based serum 112 may be buffed with a small grit side of a buffer or any other appropriate material. The buffing process buffs down loose layers of keratin from the nail plate 110 and pushes the oil-based serum 112 into the nail plate. If all of the oil-based serum 112 is absorbed into the nail plate 110 and surrounding skin, a user may choose to apply additional coats of oil-based serum 112 after the first coat of oil-based serum 112 is absorbed. The buffing stage is repeated until the nail plate 110 stops absorbing the oil-based serum 112, i.e., a sign that an appropriate amount of oil-based serum 112 has been applied. At this point, a user will begin buffing their nail plate 110 with the smoother (e.g., leather or non-grit side of a buffer). This second buffing process further pushes the remaining oil-based serum 112 into the nail plate 110 and evenly blends the oil-based serum 112, in a process similar to buffing a shoe with stain. The time required for buffing depends on the person, the condition of their nails, the amount of keratin to be removed from the nails, and the amount of oil-based serum applied and/or remaining on the nails. Again, the stage of buffing the nails is counter-intuitive to a normal manicure/pedicure process wherein the buffing process is completed on dry nail plates. The dry buffing can damage the nail plate by removing layers of keratin, scratching the nail plate, thinning the nail plate, etc. Here, the buffing of nails coated in an oil-based serum prevents the damage of nail plate and, in fact, improves the nail plate and helps to restore its natural balance of oils and water, while removing loose keratin.

After the oil-based serum coat(s) 112 has been absorbed, a coat of base composition 116 may be applied directly to the oil-based serum coat 112. The base composition 116 may further “push” the oil-based serum into the nail plate, thereby strengthening its attachment into the nail plate 110 and its crevices 114. The filling of the crevices allows for a smooth nail plate surface to apply the next layers of products. The coat of base composition 116 may be applied in any appropriate amount, enough to completely cover the oil-based serum coat on the nail. Drying time of the base composition 116 may vary based on various factors, including, but not limited to, the thickness of the coat of base composition, the conditions and temperature in the location of the nail coating composition process, etc. The base composition 116 may be dried by exposure to the environment or the finger 100 may be placed in a dryer and/or heat lamp to accelerate drying time, e.g., a UV light or an LED light. A user may choose to apply a second coat of base composition after the first coat of base composition has dried, for example, if they do not believe it has been evenly coated.

After the base composition 116 has dried, a coat of UV/LED composition 118 may be applied directly to the base composition coat 116. The coat of UV/LED composition 118 may be applied in any appropriate amount, enough to completely cover the base composition coat 116 on the nail plate 110. Drying time of the UV/LED composition 118 may vary based on various factors, including, but not limited to, the thickness of the coat of UV/LED composition 118, the conditions and temperature in the location of the nail coating composition process, etc. The UV/LED composition coat 118 may be dried by placing the finger or toe 100 in a dryer and/or heat lamp to accelerate drying time, e.g., a UV light or an LED light. The UV or LED light activates the layer and allows it to cure and dry. A user may choose to apply a second coat of UV/LED composition 118 after the first coat of UV/LED composition has dried, if they do not believe it has been evenly coated.

The nail coating system can be limited to the steps above. The resulting coated nail plates have several unexpected resulting benefits. First, the end product is a high quality coated nail plate. The nail plate restores its natural sebum and water, therefore resulting in improved nail health such as improved strength and a less brittle, dry nail than those treated in other nail treatment systems. Second, the nail coating system is healthier to be around without the use of various chemicals that strip the nail plate, including, formaldehyde, alcohol, and acetone. Third, the coated nail plate exhibits a smooth nail plate surface. Any additional products applied to the smooth, coated nail plate will have improved adhesion and be less likely to chip and/or bubble at weakened points around bumps and crevices, resulting in a longer-lasting nail polish wear. These results are unexpected as the normal manicure/pedicure routine involves removing the natural sebum and water from a nail plate in order to create a better adhesion of additional cosmetic nail products. Further, the results are unexpected because nail polish would not be able to adhere to a nail plate having been treated with oil such as the oil-based serum of the present nail coating system. Fourth, the nail plates treated by the present nail coating system may be better protected and less prone to breaking and damage as a bare nail. Fifth, the nail plates treated by the present nail coating system may grow out faster since they are less likely to become damaged, e.g., less cracking, splitting, peeling, etc., due to their improved strength, flexibility, hydration, and balanced sebum and water levels. This is also unique as compared to current systems which can either strength/harden nail plates or moisturize the nails. The current systems cannot do both at the same time—rather, the current systems switch back and forth between strengthening and moisturizing the nail plates—resulting in nail plates that are lacking at least some of the benefits of the present nail coating system at any given moment. Moreover, many current systems are not able to be combined with nail polish or other manicure systems.

In an embodiment, shown in FIG. 2, a nail plate 210 may be treated with a nail coating system 200 through the application of oil-based serum 212, followed by buffing and the application of additional coats of oil-based serum 212, filing the nail plate crevices 214 and creating a smooth nail plate 210 surface free of loose keratin and restored to its natural balance of sebum and water. A coat of base composition 216 may be applied on top of the oil-based serum 212 coat. After the base composition coat 216 dried, a coat of UV/LED composition 218 may be applied on top of the base composition 216 and cured under a heat lamp or light.

An optional coating of standard nail polish 220, e.g., colored nail lacquer, textured nail lacquer, etc. or any other traditional solvent based lacquer, may be applied to the dried coat of base composition 218. This provides a pop of color or texture to a finger or toe. All of the nail plates 210 may feature the same color or colors of nail polish 220, or one nail plate may feature different colors or patterns than another nail plate. In this embodiment, nail polish remover (not shown), e.g., acetone or a non-acetone solvent, may be used to remove the coating of standard nail polish 220 when the user would like to change, update or remove the color, e.g., when the nail grows. An unexpected benefit of the nail coating system 200 is that the nail polish remover can remove the standard nail polish coating 220 without removing the UV/LED component 218, the base component coating 216 or the oil-based serum 212. Accordingly, the ability to use a nail polish remover to remove the polish coating and enable a reapplication of a new coating without altering the integrity of the base. This allows users to gain flexibility in switching colors of nail polish without losing the durability or longevity of the UV/LED component coating, the base component coating and the oil-based serum coatings that are providing treatment and protection to the nail plate. This embodiment of a nail coating composition system has never been done in the marketplace and provides unexpected results and benefits to the user.

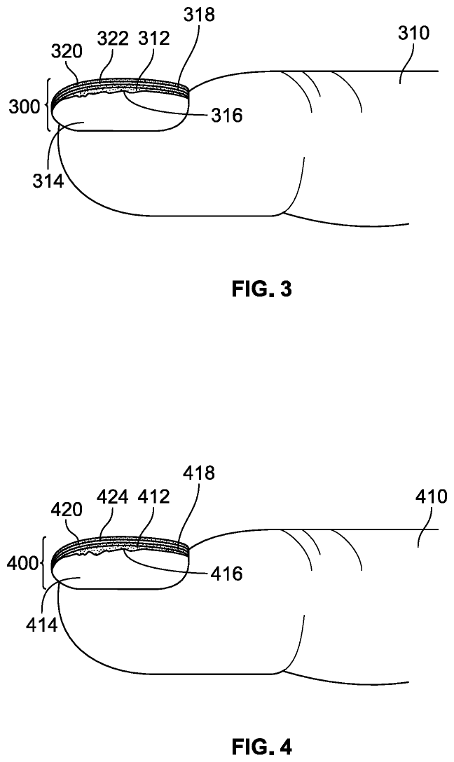

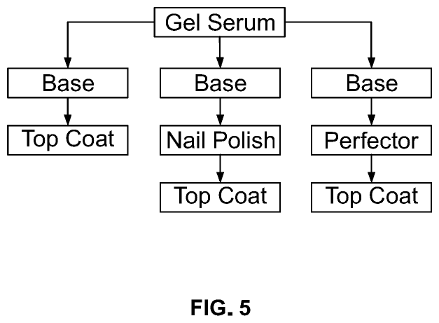

In an embodiment, shown in FIG. 3, a nail plate 310 may be treated with a nail coating system 300 through the application of oil-based serum 312, followed by buffing and the application of additional coats of oil-based serum 312, filing the nail plate crevices 314 and creating a smooth nail plate 310 surface free of loose keratin and restored to its natural balance of sebum and water. A coat of base composition 316 may be applied on top of the oil-based serum 312 coat. After the base composition coat 316 dried, a coat of UV/LED composition 318 may be applied on top of the base composition 316 and cured under a heat lamp or light. An optional coating of standard nail polish 320, e.g., colored nail lacquer, textured nail lacquer, etc. or any other traditional solvent based lacquer, may be applied to the dried coat of base composition 318. This provides a pop of color or texture to a finger or toe. All of the nail plates 310 may feature the same color or colors of nail polish 320, or one nail plate may feature different colors or patterns than another nail plate. An optional coating of traditional top coat 322, e.g., a clear nail lacquer, may be applied to the dried standard nail polish coating 320. The application of an optional coating of traditional top coat allows for further sealing of the nail coating system, and provides a shiny, manicured finish on the nail plate. This embodiment of a nail coating composition system has never been done in the marketplace and provides unexpected results and benefits to the user.

In this embodiment, nail polish remover (not shown), e.g., acetone or a non-acetone solvent, may be used to remove the coating of traditional top coat 322 and standard nail polish 320 when the user would like to change, update or remove the color, e.g., when the nail grows. An unexpected benefit of the nail coating system 300 is that the nail polish remover can remove the standard nail polish coating 320 without removing the UV/LED component 318, the base component coating 316 or the oil-based serum 312. Accordingly, the ability to use a nail polish remover to remove the polish coating and enable a reapplication of a new coating without altering the integrity of the base. This allows users to gain flexibility in switching colors of nail polish without losing the durability or longevity of the UV/LED component coating, the base component coating and the oil-based serum coatings that are providing treatment and protection to the nail plate.

In an embodiment, shown in FIG. 4, a nail plate 410 may be treated with a nail coating system 400 through the application of oil-based serum 412, followed by buffing and the application of additional coats of oil-based serum 412, filing the nail plate crevices 414 and creating a smooth nail plate 410 surface free of loose keratin and restored to its natural balance of sebum and water. A coat of base composition 416 may be applied on top of the oil-based serum coat 412. After the base composition coat 416 dried, a coat of UV/LED composition 418 may be applied on top of the base composition 416 and cured under a heat lamp or light. An optional coating of standard nail polish 420, e.g., colored nail lacquer, textured nail lacquer, etc. or any other traditional solvent based lacquer, may be applied to the dried coat of base composition 418. This provides a pop of color or texture to a finger or toe nail plate. All of the nail plates 410 may feature the same color or colors of nail polish 420, or one nail plate may feature different colors or patterns than another nail plate. An optional coating of traditional top coat 422, e.g., a clear nail lacquer, may be applied to the dried standard nail polish coating 420. An optional second UV/LED composition coat 424 may be applied to a dried traditional top coat 422 or to a dried standard nail polish coating 420 and cured under a heat lamp or light. This may allow for longer wear. The application of the optional second UV/LED coat 424 may negate the functionality of using nail polish remover to remove only the layers of standard nail polish 420 and traditional top coat 422, as the removal of the second UV/LED coat 424 could result in removing all of the layers of the nail coating system.

While the technology has been described with reference to various exemplary embodiments, it will be appreciated that the modifications may occur to those skilled in the art, and the present application is intended to cover such modifications and invention as fall within the spirit of the invention.

What is claimed is:

1. A nail coating system comprising:
(a) an oil-based serum;
(b) a base composition; and
(c) a UV/LED composition.

2. The nail coating system of claim 1, wherein the base composition comprises a material selected from the group consisting of: acetate, butyl acetate, or derivatives thereof.

3. The nail coating system of claim 1, wherein a coating of oil-based serum is applied directly to a clean nail plate.

4. The nail coating system of claim 3, wherein the oil-based serum coating restores sebum and water to the nail plate.

5. The nail coating system of claim 4, wherein the oil-based serum fills crevices in the nail plate.

6. The nail coating system of claim 5, wherein a coating of base composition is applied to the oil-based serum coating.

7. The nail coating system of claim 1 further comprising a nail polish coating.

8. The nail coating system of claim 1 further comprising a top coat.

9. The nail coating system of claim 1 further comprising a second UV/LED composition.

10. A method of using a nail coating composition comprising the steps of:
(a) applying an oil-based serum coating to a nail plate;
(b) buffing the oil-based serum coating into the nail plate;
(c) applying a base composition coating to the oil-based serum coating; and
(d) applying a UV/LED composition coating to the base composition coating.

11. The method of claim 10, further comprising:
applying a nail polish coating to the UV/LED composition coating.

12. The method of claim 11, further comprising:
applying a top coat coating to the nail polish coating.

13. The method of claim 12, further comprising:
applying a second UV/LED composition coating to the top coat coating.

14. The method of claim 11, further removing the nail polish coating with a nail polish remover and maintaining the UV/LED composition coating, the base composition coating, and the oil-based serum coating.

15. The method of claim 14, further applying a new nail polish coating to the UV/LED composition coating.

16. The method of claim 11, further removing the top coat coating and the nail polish coating with a nail polish remover and maintain the UV/LED composition coating, the base composition coating and the oil-based serum coating.

17. The method of claim 16, further applying a new nail polish coating to the UV/LED composition coating.

18. A method of using a nail coating composition comprising the steps of:
(a) applying an oil-based serum coating to a nail plate with crevices;
(b) filling the crevices of the nail plate with the oil-based serum;
(c) applying a base composition coating to the oil-based serum coating;
(d) pushing the oil-based serum into the nail plate with the base composition; and
(e) applying a UV/LED composition coating to the base composition coating.

19. The method of claim 18, wherein the coated nail plate has restored sebum and water levels.

20. The method of claim 18, wherein the coated nail plate has improved nail health.
